(19) United States
(12) Patent Application Publication
(10) Pub. No.: US 2025/0143557 A1
(43) Pub. Date: May 8, 2025

(54) URETEROSCOPES WITH LASER FIBER CHANNEL

(71) Applicant: C.R. BARD, INC., Murray Hill, NJ (US)

(72) Inventors: Cecille Canary, Atlanta, GA (US); Justin Wolfe, Lawrenceville, GA (US); Paul Thomas Mannion, Eliot, ME (US)

(21) Appl. No.: 18/835,937

(22) PCT Filed: Feb. 9, 2022
(86) PCT No.: PCT/US2022/015828

§ 371 Date: Aug. 5, 2024

(51) Int. Cl. A61B 1/07; A61B 1/012

(52) U.S. Cl. CPC A61B 1/07; A61B 1/012

(57) ABSTRACT

An example, ureteroscope includes a handpiece at least defining a working channel port and a laser fiber port. The ureteroscope also includes a catheter including a proximal end and a distal end opposite the proximal end. The catheter at least defines a working channel and a laser fiber channel that is distinct from the working channel. In other word, the catheter defines a dedicated laser fiber channel that is distinct from the working channel. The working channel and the laser fiber channel are connected (either directly or indirectly) to the working channel port and the laser fiber port, respectively.

### BACKGROUND

Endoscopes of small size are desired in many industrial and medical applications. For example, when natural orifices and lumens of a human body are small, small endoscopes are used for insertion through such orifices and lumens to target locations within the body. For single incision laparoscopy, smaller endoscopes are preferred to provide an inside-the-body view of the surgical site, particularly when the incision itself is of minimal dimensions. Sometimes, patients may feel irritation when an endoscope is being inserted into his or her body, and a smaller endoscope may mitigate such unpleasant experience and may minimize trauma to the patient. Moreover, a physician may improve diagnostic and procedural protocols with a smaller endoscope. For example, transnasal endoscopy may sometimes replace trans-oral endoscopy.

### SUMMARY

In an embodiment, a ureteroscope is disclosed. The ureteroscope includes a handpiece at least defining a working channel port and a laser fiber port. The ureteroscope also includes a catheter including a proximal end and a distal end opposite the proximal end. The catheter at least defines a working channel extending from the proximal end to the distal end of the catheter and a laser fiber channel extending from the proximal end to the distal end of the catheter. The laser fiber channel is distinct from the working channel. The working channel is connected to the working channel port and the laser fiber channel is connected to the laser fiber port. The ureteroscope also includes a laser fiber. At least one of the laser fiber port includes a funnel structure exhibiting a lateral dimension that decreases with increasing distance from an exterior of the handpiece, an optional fiber conduit extending from the laser fiber port to the laser fiber channel includes a funnel structure exhibiting a lateral dimension that decreases with increasing distance from an exterior of the handpiece, or the laser fiber is pre-loaded into the laser fiber channel.

In an embodiment, a method of using an ureteroscope is disclosed. The method includes inserting a distal end of a catheter of the ureteroscope into a urethral opening of an individual. The ureteroscope includes a handpiece at least defining a working channel port and a laser fiber port. The ureteroscope also includes a catheter including a proximal end and a distal end opposite the proximal end. The catheter at least defines a working channel extending from the proximal end to the distal end of the catheter and a laser fiber channel extending from the proximal end to the distal end of the catheter. The laser fiber channel is distinct from the working channel The working channel is connected to the working channel port and the laser fiber channel is connected to the laser fiber port. The ureteroscope also includes a laser fiber. At least one of the laser fiber port includes a funnel structure exhibiting a lateral dimension that decreases with increasing distance from an exterior of the handpiece, an optional fiber conduit extending from the laser fiber port to the laser fiber channel includes a funnel structure exhibiting a lateral dimension that decreases with increasing distance from an exterior of the handpiece, or the laser fiber is pre-loaded into the laser fiber channel.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a side elevational and schematic view of a endoscopic system, according to an embodiment.

FIG. 1B is a cross-sectional view of a portion of the ureteroscope taken along plane 1B-1B, according to an embodiment.

FIG. 1C is a side elevational view of the distal end of the catheter, according to an embodiment.

FIG. 2 is a cross-sectional schematic of a ureteroscope, according to an embodiment.

FIG. 3 is a cross-sectional view of a ureteroscope, according to an embodiment.

FIG. 4 is a cross-sectional view of a ureteroscope, according to an embodiment.

FIG. 5 is a flow diagram of a method of operating any of the ureteroscopes disclosed herein.

### DETAILED DESCRIPTION

Embodiments are directed to ureteroscopes including a dedicated laser fiber channel and methods of using the same. An example, ureteroscope includes a handpiece at least defining a working channel port and a laser fiber port. The ureteroscope also includes a catheter including a proximal end and a distal end opposite the proximal end. The catheter at least defines a working channel and a laser fiber channel that is distinct from the working channel. In other word, the catheter defines a dedicated laser fiber channel that is distinct from the working channel. The working channel and the laser fiber channel are connected (either directly or indirectly) to the working channel port and the laser fiber port, respectively.

The functionality of the ureteroscope is improved relative to conventional ureteroscopes. For example, conventional ureteroscopes include a catheter defining a working channel. Conventionally, the laser fiber is disposed in the working channel. Disposing the laser fiber in the working channel decreases the space in the working channel to receive surgical instruments. As such, disposing the laser fiber in the working channel may require inserting and removing the laser fiber from the working channel depending on which surgical instruments are inserted into the working channel. Removing the laser fiber from the working channel limits when the laser fiber may be used. Also, inserting the laser fiber into the working channel may be difficult since working channels often include bends therein and the laser fiber may have difficultly moving through the bend, especially when the laser fiber exhibits a diameter less than 500 μm since such laser fibers may buckle rather than traverse the bend. It is noted that buckling the laser fibers may damage the laser fibers and may affect the ability of a laser to be reflected within the laser fiber. Further, the laser fiber may become caught (e.g., snagged) on the surgical instruments disposed in the working channel while the laser fiber is inserted into the working channel which may inhibit further insertion of the laser fiber into the working channel. Additionally, it has been found that laser fibers may move in the working channel. For example, emitting a laser from a distal tip of the laser fibers and shockwaves caused by irradiating an object (e.g., kidney stone) with the emitted laser may cause non-negligible movement (e.g., lateral movement) at the distal portion of the laser fiber thereby reducing the accuracy of the laser fiber.

The ureteroscopes disclosed herein remedy these deficiencies of conventional ureteroscopes by including a dedicated laser fiber channel that is distinct from the working channel. The laser fiber channel may be at least partially formed in portions of the catheter that would otherwise be occupied to a body of the catheter or by unoccupied space. As such, the inclusion of the laser fiber channel may have no or negligible effect on the overall size of the catheter or the size of the working channel of the catheter. The laser fiber channel allows the laser fiber to remain disposed in the catheter thereby negating the need to remove or insert the laser fiber into the catheter to accommodate different surgical instruments. The laser fiber channel is also configured to receive the laser fiber thereby preventing movement of the laser fiber caused by the laser fiber emitting a laser or shockwaves. The laser fiber channel also facilitates insertion and movement of the laser fiber when such insertion and movement is needed. For example, the laser fiber channel may include one or more features (e.g., introducer element, funnel structure, actuators, etc.) configured to facilitate inserting the laser fiber into the laser fiber channel and moving the laser fiber through a bend in the laser fiber channel. Also, the laser fiber channel may not include other components (e.g., surgical components) therein on which the laser fiber may be caught on.

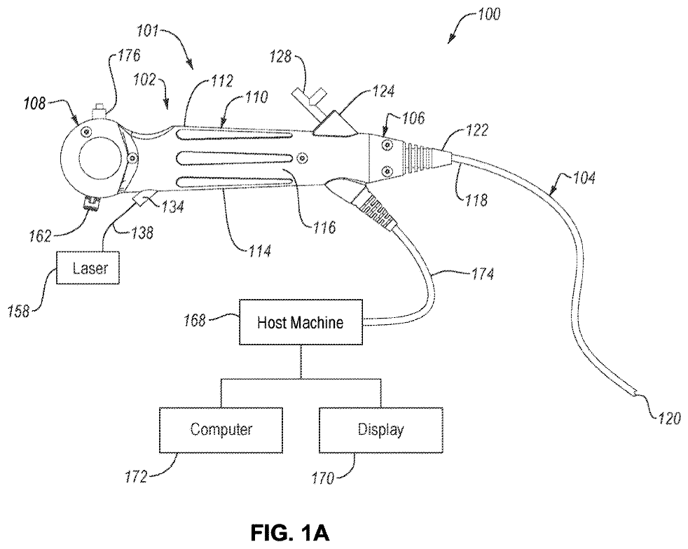

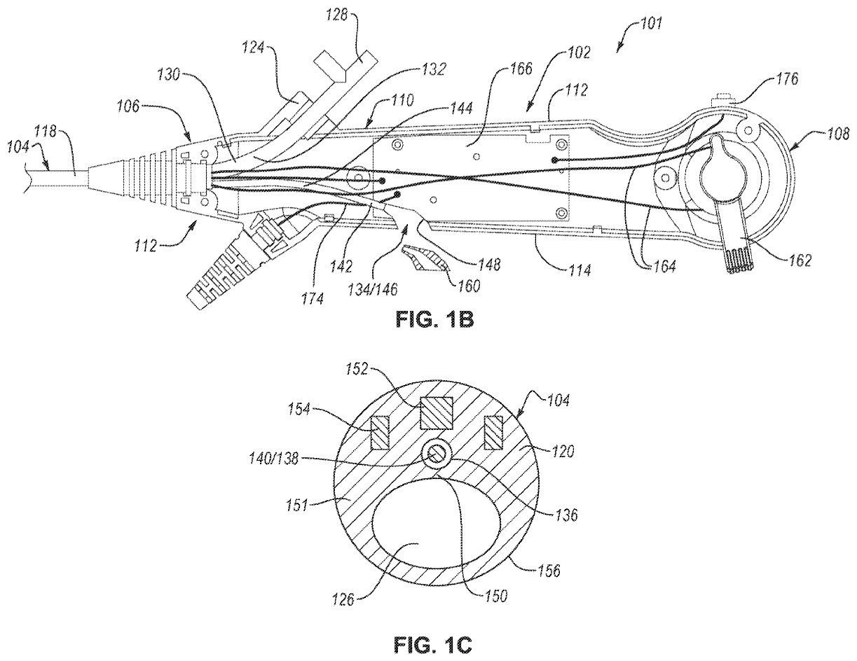

FIG. 1A is a side elevational and schematic view of a endoscopic system 100, according to an embodiment. The endoscopic system 100 includes a ureteroscope 101. FIG. 1B is a cross-sectional view of a portion of the ureteroscope 101 taken along plane 1B-1B, according to an embodiment. The ureteroscope 101 includes a handpiece 102 and a catheter 104. The handpiece 102 is attached to or integrally formed with the catheter 104. The catheter 104 extends from the handpiece 102.

The handpiece 102 is elongated and includes a catheter end 106 and a control end 108. The catheter end 106 is proximate to the catheter 104 and a control end 108 opposite or distal to the catheter end 106. As the control end 108 is opposite to the catheter end 106, the control end 108 and the catheter end 106 are at different ends of the handpiece 102 and may face different directions relative to one another. The control end 108 may include a generally bulbous shape, and the handpiece 102 may taper between the control end 108 and the catheter end 106.

With the control end 108 opposite or distal to the catheter end 106, the handpiece 102 includes an intermediate or central portion 110 positioned between the catheter end 106 and the control end 108. The central portion 110 includes a first or top surface 112, a second or bottom surface 114, and two opposing sides 116 positioned between the top surface 112 and the bottom surface 114. In an embodiment, the top and bottom surfaces 112, 114 of the central portion 110 are rounded and the sides 116 are substantially flat.

In an embodiment, both the catheter 104 and the handpiece 102 are disposable. In some embodiments, the catheter 104 and the handpiece 102 are manufactured as an integral part, or the catheter 104 is fixed with the handpiece 102 via a handpiece-catheter connector 122. Alternatively, only the catheter 104 is disposable, and the handpiece 102 may be sterilized and reused multiple times. In this case, the catheter 104 is removably connected to the handpiece 102 and the catheter end 106 via a handpiece-catheter connector 122.

The catheter 104 includes a proximal end 118 and a distal end 120 spaced from the proximal end 118. The proximal end 118 is attached to, integrally formed with, or disposed in the catheter end 106 (e.g., the handpiece-catheter connector 122). The distal end 120 is configured to be disposed in the urethral opening of an individual. FIG. 1C is a side elevational view of the distal end 120 of the catheter 104, according to an embodiment.

The handpiece 102 defines a working channel port 124 and the catheter 104 defines a working channel 126 (FIG. 1C). The working channel 126 extends from the proximal end 118 to the distal end 120 of the catheter 104. The working channel port 124 and the working channel 126 are connected together (either directly or indirectly). The working channel port 124 and the working channel 126 are configured to engage with various surgical instruments and irrigation devices, as needed, for operations such as stone breaking and retrieval, etc. For example, the various surgical instruments may be inserted through the working channel port 124 and through the working channel 126 such that the various surgical instruments may be used at the distal end 120 of the catheter 104. The handpiece 102 may also include at least one of a working channel connector 128 that is connected or attached to the working channel port 124 or at least one working conduit 130 that indirectly connects the working channel port 124 to the working channel 126.

The working channel port 124 may be positioned proximate to the catheter end 106 of the handpiece 102. For example, the working channel port 124 may be positioned less than one-half of a distance from the catheter end 106 to the control end 108, less than one-third of the distance from the catheter end 106 to the control end 108, less than one-quarter of the distance from the catheter end 106 to the control end 108, or less than one-fifth of the distance from the catheter end 106 to control end 108. Positioning the working channel port 124 proximate to the catheter end 106 may improve the ergonomics of the handpiece 102 since the working channel port 124 and any surgical instruments extending therefrom are less likely to interfere with use of the ureteroscope 101. Positioning the working channel port 124 proximate to the catheter end 106 may cause at least one of the working channel port 124, a portion of the working channel 126 disposed within the catheter end 106, or the optionally working conduit 130 to exhibit a working bend 132 therein when the catheter 104 is substantially straight. Due to the proximity of the working channel port 124 to the catheter end 106, the working bend 132 may exhibit a radius of curvature that is less than 10 cm. As used herein, radius of curvature refers to at least one of the radius of curvature of a single point of a bend (e.g., the working bend 132 or the fiber bend 144), the average radius of curvature along a portion of a bend, or the average radius of curvature along an entire bend.

The handpiece 102 defines a laser fiber port 134 and the catheter 104 defines a laser fiber channel 136 (FIG. 1C). The laser fiber channel 136 extends from the proximal end 118 to the distal end 120 of the catheter 104. The laser fiber port 134 and the laser fiber channel 136 are connected together (either directly or indirectly). The laser fiber port 134 and the laser fiber channel 136 are configured to receive and engage with a laser fiber 138. For example, the laser fiber 138 may be inserted through the laser fiber port 134 and through the laser fiber channel 136 such that a distal portion 140 of the laser fiber 138 is positioned at the distal end 120 of the catheter 104. A laser may be emitted from the distal portion 140 of the laser fiber 138 towards an object (e.g., kidney stone) that is adjacent to or proximate to the distal end 120 of the catheter 104. The handpiece 102 may optionally include at least one fiber conduit 142 that indirectly connects the laser fiber port 134 to the laser fiber channel 136.

The working channel port 124 and the laser fiber port 134 are openings formed in the handpiece 102 that are connected, either directly or indirectly, to the working channel 126 and the laser fiber channel 136, respectively. In an embodiment, the working channel port 124 and the laser fiber port 134 are distinct from each other. The working channel port 124 and the laser fiber port 134 are distinct from each other when the openings of the working channel port 124 and the laser fiber port 134 are spaced from each other. In an embodiment, the working channel port 124 and the laser fiber port 134 are not distinct from each other. In other words, the working channel port 124 and the laser fiber port 134 share the same opening in the handpiece 102. When the working channel port 124 and the laser fiber port 134 are not distinct, the working channel 126 and the laser fiber channel 136 extend from the same opening in the handpiece 102.

When the working channel port 124 and the laser fiber port 134 are distinct from each other, the laser fiber port 134 may be located on any portion of the handpiece 102, without limitation. For example, the laser fiber port 134 may be positioned less than one-half of a distance from the catheter end 106 to the control end 108, less than one-third of the distance from the catheter end 106 to the control end 108, less than one-quarter of the distance from the catheter end 106 to the control end 108, less than one-fifth of the distance from the catheter end 106 to control end 108, a midpoint in the handpiece 102 (e.g., a midpoint of the central portion 110), less than one-half of a distance from the control end 108 to the catheter end 106, less than one-third of the distance from the control end 108 to the catheter end 106, less than one-quarter of the distance from the control end 108 to the catheter end 106, less than one-fifth of the distance from the control end 108 to catheter end 106, or at the control end 108. In an example, the working channel port 124 and the laser fiber port 134 are formed in the same general location (e.g., within about 2 cm) from each other. The surgical instruments and the laser fiber 138 extend outwardly from the same general location of the handpiece 102 when the working channel port 124 and the laser fiber port 134 have the same general location. Having the surgical instruments and the laser fiber 138 extend from the same general location on the handpiece 102 improves the ergonomics of the ureteroscope, for instance, by allowing additional portions of the handpiece 102 to be gripped without interfering with the surgical instruments and the laser fiber 138 than if the working channel port 124 and the laser fiber port 134 where not in the same general location. In an example, the working channel port 124 and the laser fiber port 134 are positioned on different surfaces of the handpiece 102 and are located substantially the same general distance from the catheter end 106 of the handpiece 102. For instance, the working channel port 124 may be positioned on the top surface 112 and the laser fiber port 134 may be positioned on the bottom surface 114 or vice versa or the working channel port 124 and the laser fiber port 134 may be located on different side surfaces 116. Locating the working channel port 124 and the laser fiber port 134 substantially the same distance from the catheter end 106 improves the ergonomics of the ureterscope, for instance, by allowing additional portions of the handpiece 102 to be gripped without interfering with the surgical instruments and the laser fiber 138 than if the laser fiber port 134 was located further or closer to the catheter end 106 than the working channel port 124. Further, locating the working channel port 124 and the laser fiber port 134 on different surfaces of the handpiece 102 decreasing the likelihood that manipulating the surgical instruments (e.g., inserting, removing, or otherwise moving the surgical instruments) causes movement in the laser fiber 138 or vice versa. In an example, the laser fiber port 134 is spaced closer to or, more particularly, further from the catheter end 106 than the working channel port 124. Spacing the laser fiber port 134 further from the catheter end 106 than the working channel port 124 causes at least one of the laser fiber port 134, the laser fiber channel 136, or the optional fiber conduit 142 to exhibit a bend 144 (hereinafter referred to as “fiber bend”) when the catheter 104 is substantially straight. The fiber bend 144 may exhibit a radius of curvature that is greater than the working bend 132. The likelihood that the laser fiber 138 becomes caught (e.g., snagged) on the walls defining the fiber bend 144 or buckles decreases as the radius of curvature increases. Thus, positioning the laser fiber port 134 further from the catheter end 106 decreases the likelihood that the laser fiber 138 may become caught on the walls defining the fiber bend 144 and that the laser fiber 138 buckles when inserting the laser fiber 138 through the fiber bend 144.

The laser fiber port 134 or the optional fiber conduit 142 includes a funnel structure 146. The funnel structure 146 exhibits a lateral dimension that decreases with increasing distance from an exterior surface (e.g., the top, bottom, or side surfaces 112, 114, 116) of the handpiece 102. In effect, the funnel structure 146 increases the size of the opening of the laser fiber port 134 at the exterior of the handpiece 102 and decreases size of the laser fiber channel 136. The increased size of the opening of the laser fiber port 134 caused by the funnel structure 146 facilitates insertion of the laser fiber 138 into the laser fiber port 134. For example, as will be discussed in more detail below, the laser fiber 138 may exhibit a relatively small maximum lateral dimension (e.g., about 50 μm to 500 μm). A person attempting the insert the laser fiber 138 into the laser fiber port 134 may find such insertion difficult if the laser fiber port 134 exhibited a lateral dimension that was comparable to the maximum lateral dimension of the laser fiber 138. Thus, the increased size of the laser fiber port 134 caused by the funnel structure 146 makes facilitates insertion of the laser fiber 138 into the laser fiber port 134. The decreased size of the laser fiber channel 136 relative to the laser fiber port 134, as caused by the funnel structure 146, prevents or at least inhibits back flow of bodily fluids (e.g., urine) from the distal end 120 of the catheter 104 to the laser fiber port 134. For example, as will be discussed in more detail below, there may be a gap between the walls defining the laser fiber channel 136 and the laser fiber 138 disposed therein. Bodily fluids may flow through the gap. Decreasing the size of the laser fiber channel 136 decreases the size of the gap through which bodily fluids may flow which, in turn, decreases or prevents bodily fluids flow through the gap.

The funnel structure 146 is defined by one or more side walls 148. The one or more side walls 148 may be tapered or curved (e.g., concavely or convexly curved relative to the exterior of the handpiece 102). In other words, the side walls 148 may not be stepped since the stepped side walls 148 may cause the laser fiber 138 to become caught thereon when the laser fiber 138 is inserted into the funnel structure 146. Catching the laser fiber 138 may prevent further insertion of the laser fiber 138 into the funnel structure 146 without buckling the laser fiber 138.

The laser fiber channel 136 is configured to have the laser fiber 138 disposed therein. The working channel 126 and the laser fiber channel 136 are distinct from each other. The working channel 126 and the laser fiber channel 136 are distinct from each other when object(s) (e.g., surgical instruments or the laser fiber 138) positioned in one channel cannot enter the other channel. For example, the working channel 126 and the laser fiber channel 136 are distinct when there is a barrier 150 between the working channel 126 and the laser fiber channel 136. In an embodiment, as illustrated in FIG. 1C, the catheter 104 may include a body 151 that defines both the working channel 126 and the laser fiber channel 136. In such an embodiment, the portion of the body 151 between the working channel 126 and the laser fiber channel 136 forms the barrier 150. In an embodiment, the catheter 104 is a large tube and the working channel 126 and the laser fiber channel 136 are smaller tubes that are disposed in the interior of the large tube. In such an embodiment, the walls of the smaller tubes that form the working channel 126 and the laser fiber channel 136 form the barrier 150.

The laser fiber channel 136 may be formed in portions of the catheter 104 that would otherwise be occupied by the body 151 or is unoccupied space. Forming the laser fiber channel 136 in portions of the catheter 104 that would otherwise be occupied by the body 151 or is unoccupied space prevents or at least minimizes any increase in the size of the catheter 104 due to including the laser fiber channel 136 in the catheter 104. The laser fiber channel 136 may be formed in portions of the catheter 104 that would otherwise be occupied by the body 151 or is unoccupied space due to the relatively small size of the laser fiber 138, especially when the laser fiber 138 is a thulium laser fiber (i.e., a laser fiber configured to be used with a thulium laser). In an embodiment, as illustrated, the laser fiber channel 136 may be located between the working channel 126 and a optoelectronic module 152 when there is sufficient spaced between the working channel 126 and the optoelectronic module 152 for the laser fiber channel 136. In an embodiment, the laser fiber channel 136 may be located between the working channel 126 and the light source 154, between the working channel 126 and an exterior 156 of the catheter 104, between the optoelectronic module 152 and the light source 154, between the optoelectronic module 152 and the exterior 156 of catheter 104, between the light source 154 and the exterior 156 of the catheter 104, or any other location on the catheter 104 depending on where there is sufficient space to form the laser fiber channel 136.

In an embodiment, the working channel 126, the laser fiber channel 136, and the optoelectronic module 152 are aligned on the same axis. Aligning the working channel 126, the laser fiber channel 136, and the optoelectronic module 152 may facilitate using the ureteroscope 101 because the laser emitted from the laser fiber channel 136 and the surgical instruments protruding from the working channel 126 are aligned on the display 170 of the endoscopic system 100. In an embodiment, the laser fiber channel 136 may not be aligned on the same axis as the working channel 126 and the optoelectronic module 152.

As previously discussed, at least one of the laser fiber port 134, the laser fiber channel 136, or the optional fiber conduit 142 includes the fiber bend 144 when the catheter 104 is substantially straight. The laser fiber 138 may resist bending as the laser fiber 138 is inserted into and moves through the fiber bend 144 which may cause the laser fiber 138 to become caught on the walls defining the fiber bend 144. For example, the laser fiber 138 may become caught on the walls defining the fiber bend 144 due to friction between the wall and the laser fiber 138 or the distal portion 140 of the laser fiber 138 at least partially penetrates or otherwise becomes snagged on the walls defining the fiber bend 144. Forcing the laser fiber 138 through the fiber bend 144 while the laser fiber 138 is caught on the walls defining the fiber bend 144 may cause the laser fiber 138 to buckle, especially as the maximum lateral dimension is decreased. Generally, increasing the radius of curvature of the fiber bend 144 makes it easier for the laser fiber 138 to be inserted into and move through the fiber bend 144 without the laser fiber 138 becoming caught on the walls defining the fiber bend 144. In an embodiment, the radius of curvature of the fiber bend 144 may be selected to be greater than the radius of curvature of the working bend 132 which may allow the laser fiber 138 inserted into and moved through the fiber bend 144 easier than the working bend 132. The radius of curvature of the fiber bend 144 may be greater than the radius of curvature of the working bend 132 when the laser fiber port 134 is spaced further from the catheter end 106 than the working channel port 124.

In an embodiment, the radius of curvature of the fiber bend 144 may be selected to be about 7.5 cm or greater, about 10 cm or greater, about 12.5 cm or greater, about 15 cm or greater, about 17.5 cm or greater, about 20 cm or greater, about 22.5 cm or greater, about 25 cm or greater, about 30 cm or greater, about 35 cm or greater, about 40 cm or greater, about 45 cm or greater, about 50 cm or greater, or in ranges of about 7.5 cm to about 12.5 cm, about 10 cm to about 15 cm, about 12.5 cm to about 17.5 cm, about 15 cm to about 20 cm, about 17.5 cm to about 22.5 cm, about 20 cm to about 25 cm, about 22.5 cm to about 30 cm, about 25 cm to about 35 cm, about 30 cm to about 40 cm, about 35 cm to about 45 cm, or about 40 cm to about 50 cm. The radius of curvature of the fiber bend 144 may be selected based on a number of factors. In an example, the radius of curvature of the fiber bend 144 may be selected based on the maximum lateral dimension of the laser fiber 138. For instance, the radius of curvature of the fiber bend 144 may be increased when the maximum lateral dimension of the laser fiber 138 is decreased to inhibit buckling of the laser fiber 138. In an example, the radius of curvature of the fiber bend 144 may be selected based on the location of laser fiber port 134 on the handpiece 102.

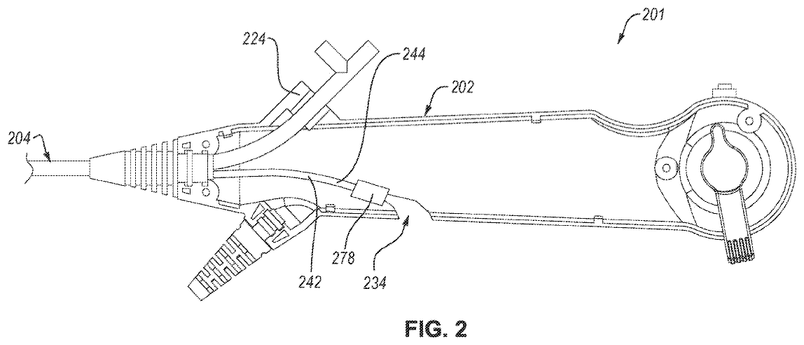

The laser fiber 138 is configured to have receive a laser at a proximal end thereof and have the laser internally reflected therein until the laser is emitted at the distal portion 140 thereof. The laser fiber 138 may be formed from any suitable material, such as silica. The laser fiber 138 may exhibit a maximum lateral dimension (e.g., diameter) that is selected to be about 1 mm or less, about 750 μm or less, about 600 μm or less, about 550 μm or less, about 500 μm or less, about 450 μm or less, about 400 μm or less, about 350 μm or less, about 300 μm or less, about 250 μm or less, about 225 μm or less, about 200 μm or less, about 175 μm or less, about 150 μm or less, about 125 μm or less, about 100 μm or less, about 75 μm or less, about 50 μm or less, about 25 μm or less, or in ranges of about 25 μm to about 75 μm, about 50 μm to about 100 μm, about 75 μm to about 125 μm, about 100 μm to about 150 μm, 125 μm to about 175 μm, about 150 μm to about 200 μm, about 175 μm to about 225 μm, about 200 μm to about 250 μm, about 225 μm to about 300 μm, about 250 μm to about 350 μm, about 300 μm to about 400 μm, about 350 μm to about 450 μm, about 400 μm to about 500 μm, about 450 μm to about 550 μm, about 500 μm to about 600 μm, about 550 μm to about 750 μm, or about 600 μm to about 1 mm. The maximum lateral dimension of the laser fiber 138 may be selected based on a number of factors. In an example, the maximum lateral dimension of the laser fiber 138 may be selected to be as small as possible thereby decreasing the size of the laser fiber channel 136 since decreasing the size of the laser fiber channel 136 makes it easier to fit the laser fiber channel 136 into the catheter 104 substantially without increasing the size of the catheter 104. In an example, the maximum lateral dimension may be selected based on the likelihood that the laser fiber 138 buckles during insertion of the laser fiber 138 into the laser fiber port 134 and laser fiber channel 136. In particular, the maximum lateral dimension of the laser fiber 138 may be increased when the likelihood that the laser fiber 138 will buckle increases. Factors that affect whether the laser fiber 138 will buckle includes the radius of curvature of the fiber bend 144 or whether the ureteroscope 101 includes an introducer element (e.g., introducer element 278 illustrated in FIG. 2). In an example, the maximum lateral dimension of the laser fiber 138 may be selected based on whether the laser fiber 138 is a thulium laser fiber (e.g., a laser fiber configured to be used with a thulium laser), a holmium laser fiber (e.g., a laser fiber configured to be used with a holmium laser), or another laser fiber. For instance, thulium laser fibers may exhibit a lateral dimension that is relatively small (e.g., less than 250 μm, less than 200 μm, or less than 150 μm) while the holmium laser fiber may exhibit a maximum lateral dimension that is relatively large (e.g., greater than 250 μm).

The laser fiber channel 136 exhibits a maximum lateral dimension (e.g., diameter) that is equal to or larger than the maximum lateral dimension of the laser fiber 138. For example, the laser fiber channel 136 may exhibit a maximum lateral dimension that is about 1.25 mm or less, about 1 mm or less, about 750 μm or less, about 600 μm or less, about 550 μm or less, about 500 μm or less, about 450 μm or less, about 400 μm or less, about 350 μm or less, about 300 μm or less, about 250 μm or less, about 225 μm or less, about 200 μm or less, about 175 μm or less, about 150 μm or less, about 125 μm or less, about 100 μm or less, about 75 μm or less, about 50 μm or less, about 25 μm or less, or in ranges of about 25 μm to about 75 μm, about 50 μm to about 100 μm, about 75 μm to about 125 μm, about 100 μm to about 150 μm, 125 μm to about 175 μm, about 150 μm to about 200 um, about 175 μm to about 225 μm, about 200 μm to about 250 μm, about 225 μm to about 300 μm, about 250 μm to about 350 μm, about 300 μm to about 400 μm, about 350 μm to about 450 μm, about 400 μm to about 500 μm, about 450 μm to about 550 μm, about 500 μm to about 600 μm, about 550 μm to about 750 μm, about 600 μm to about 1 mm, or about 750 μm to about 1.25 mm. The maximum lateral dimension of the laser fiber channel 136 is generally selected based on the maximum lateral dimension or range of maximum lateral dimensions of the laser fibers that the laser fiber channel 136 is configured to receive.

In an embodiment, the maximum lateral dimension of the laser fiber channel 136 is selected to be larger than the maximum lateral dimension of the laser fiber 138. In such an embodiment, there is a gap between the walls defining the laser fiber channel 136 and the laser fiber 138. The gap decreases the frictional forces applied to the laser fiber 138 from the walls defining the laser fiber channel 136 during insertion of the laser fiber 138 which decreases the likelihood that the laser fiber 138 buckles during insertion. The gap may exhibit a maximum lateral dimension (i.e., the maximum lateral dimension of the laser fiber channel 136 minus the maximum lateral dimension of the laser fiber 138) that is selected to be about 5 μm to about 15 μm, about 10 μm to about 20 μm, about 15 μm to about 25 μm, about 20 μm to about 30 μm, about 25 μm to about 35 μm, about 30 μm to about 40 μm, about 35 μm to about 45 μm, about 40 μm to about 50 μm, about 45 μm to about 60 μm, about 50 μm to about 70 μm, about 60 μm to about 80 μm, about 70 μm to about 90 μm, about 80 μm to about 100 μm, about 90 μm to about 125 μm, about 100 μm to about 150 μm, about 125 μm to about 175 μm, about 150 μm to about 200 μm, about 175 μm to about 225 μm, about 200 μm to about 250 μm, about 225 μm to about 300 μm, about 250 μm to about 350 μm, about 300 μm to about 400 μm, about 350 μm to about 450 μm, about 400 μm to about 500 μm, about 450 μm to about 600 μm, about 500 um to about 700 μm, about 600 μm to about 800 μm, about 700 μm to about 900 μm, about 800 μm to about 1 mm, or about 1 mm or greater. In an example, the maximum lateral dimension of the gap is selected to be as small as possible to prevent or at least minimize backflow of bodily fluids through the laser fiber channel 136 and to prevent lateral movement of the laser fiber 138 caused by emitted the laser and shockwaves generated by the laser.

In an embodiment, the laser fiber 138 exhibits a length that is about 0.5 m to about 1.5 m, about 1 m to about 2 m, about 1.5 m to about 3 m, about 2 m to about 4 m, about 3 m to about 5 m, about 4 m to about 6 m, about 5 m to about 7 m, about 6 m to about 8 m, about 7 m to about 9 m, about 8 m to about 10 m, or about 10 m or greater. The length of the laser fiber 138 may be selected based on the length of the catheter 104 and the expected maximum distance between the laser fiber port 134 and the laser 158.

In an embodiment, the laser fiber 138 is pre-loaded in the ureteroscope 101. The laser fiber 138 is pre-loaded in the ureteroscope 101 when the laser fiber 138 is inserted into at least the laser fiber channel 136 during the manufacturing of the ureteroscope 101 or otherwise positioned in at least the laser fiber channel 136 before the ureteroscope 101 is packaged. Pre-loading the laser fiber 138 into the ureteroscope 101 allows the laser fiber 138 to be inserted through the laser fiber port 134 and into the laser fiber channel 136 using a machine or an expert which decreases the likelihood that insertion of the laser fiber 138 into the laser fiber port 134 and the laser fiber channel 136 causes the laser fiber 138 to buckle. Also, it has been found that pre-loading the laser fiber 138 into the ureteroscope 101 makes using the ureteroscope 101 more sterile. For example, it has been found that handling a laser fiber 138 that is not pre-loaded in the ureteroscope 101 is likely to inadvertently contact one or more objects. In particular, the long length and small maximum lateral dimension of the laser fiber 138 makes handling the laser fiber 138 without inadvertently contacting an object difficult. Contacting the laser fiber 138 against an object, especially contacting the distal portion 140 of the laser fiber 138 against the object, may cause bacteria, other organisms, or other material to be deposited onto the laser fiber 138 which may then be transferred into a body (e.g., human or animal body) during use of the ureteroscope 101. However, pre-loading of the laser fiber 138 into the ureteroscope 101 causes the catheter 104 to prevent the portions of the laser fiber 138 disposed therein from contacting objects which, in turn, prevents or at least inhibits introducing bacteria, other organisms, or other material from being transferred to the laser fiber 138.

In an embodiment, the laser fiber 138 is not pre-loaded into the ureteroscope 101. In such an embodiment, the laser fiber 138 may be included in the packaging that includes the ureteroscope 101 or may be provided from another source.

In an embodiment, the handpiece 102 includes a seal 160. The seal 160 is configured to fit into or cover at least the laser fiber port 134. Fitting the seal 160 into or covering the laser fiber port 134 prevents or at least inhibits bodily fluids flowing through the laser fiber channel 136 from leaking out the laser fiber port 134. For example, the seal 160 may be a stopper, a flapper valve, a check valve, thick grease, or any other device that may cover or fit into at least the laser fiber port 134. In an embodiment, the seal 160 defines a recess, slot, or hole that is configured to receive the laser fiber 138 such that the laser fiber 138 may extend through and out of the laser fiber port 134 even when the seal 160 covers or is disposed in the laser fiber port 134. In an embodiment, the seal 160 is elastic such that the seal 160 may bend around the laser fiber 138 extending through and out of the laser fiber port 134.

The catheter 104 of the ureteroscope 101 may be used for imaging an interior surface of a tubular structure, such as a lumen in the body of human or animal. For example, the catheter 104 may be inserted via a subject's urethra to access various parts of the urinary tract. However, it should be appreciated that the ureteroscope 101 may be employed as an industrial endoscope when tubular structure is a part of an industrial apparatus, an equipment, a product, a machine, a production line, and the like. In some embodiments, the catheter 104 may serve as a tether, and may include a plurality of scale markings or fiducials that enable a physician to measure a distance traveled by optoelectronic module into the tubular structure, such as a lumen of a body. Other structure(s) may be built into the ureteroscope 101 as desired. For example, the handpiece 102 may include a steering controller 162 configured to control one or more steering wires 164 (FIG. 1B) that are connected to an active bend portion of the catheter 104 to deflect the distal end 120 to the desired location. Accordingly, a user may bend or curve the catheter 104 by moving the steering controller 162 on the handpiece 102.

The ureteroscope 101 may include an optoelectronic module 152 (e.g., a camera or other imager) for imaging the interior of the subject. For example, an optoelectronic module 152 and at least one light source 154 may be located in a distal end 120 of the catheter 104 or other location in the catheter 104. The optoelectronic module 152 may include a micro camera module having an image sensor microchip, a set of micro lenses, and a micro illumination module. Suitable optoelectronic modules are disclosed in U.S. Pat. No. 9,942,452, which is incorporated herein, in its entirety, by this reference. In some embodiments, the optoelectronic module 152 may be positioned in a rigid or semi-rigid shell-like housing at the distal end 120 configured for insertion into the tubular structure for imaging its interior surface. For example, the optoelectronic module 152 may be inserted into a patient's body through a natural body orifice, such as the mouth, nose, urethra, bladder, vagina, or anus. The ureteroscope 101 may therefore have different configurations for use as a gastrointestinal, a colonoscope, endoscopic ultrasound (EUS), endoscopic retrograde cholangiopancreatography, or other suitable application. Applications of the ureteroscope 101 include diagnostic observation associated with endometrial polyps, infertility, abnormal bleeding, and pelvic pain, and surgical procedure such as embryo growth arrest and uterine malformation etc.

The ureteroscope 101 may include a communication interface 166 generally located outside the catheter 104 for receiving the signal from an image sensor within the optoelectronic module 152. The communication interface 166 may be positioned within or adjacent to the handpiece 102. At least the optoelectronic module 152 may be coupled to the communication interface 166.

The endoscopic system 100 may include one or more electronic devices for processing and displaying the image data received from the optoelectronic module 152 of the ureteroscope 101. For example, the endoscopic system 100 may include one or more of a host machine 168 having a microprocessor, a computer 172 having a microprocessor, and a display 170. The host machine 168 may be connected to one or more terminals of the computer 172 and the display 170 for further processing and displaying the image data from the optoelectronic module 152. The host machine 168 or the computer 172 may be programmed with image processing software that takes as input the image data output from the optoelectronic module 152 of the ureteroscope 101 and generates two-or three-dimensional reconstructions of the body lumen that may be displayed on the display 170. Accordingly, a processor in at least one of the host machine 168, the computer 172, or the display 170 may be programmed with software that accepts as input a plurality of still images of an object generated by the optoelectronic module 152, and then output for display a three-dimensional rendering of the object based on the plurality of still images. The display 170 may include any suitable display, and may be configured to display a moving image (movie) or a still image collected by the image sensor of the optoelectronic module 152. Although shown in FIG. 1A as separate blocks, the host machine 168, the computer 172, and the display 170 may include a single device, two devices, three devices, or more than three devices.

The endoscopic system 100 also may include a cable 174 configured to operably couple the ureteroscope to at least one of the host machine 168, the computer 172, or the display 170. The cable 174 may electrically couple the communication interface 166 of the ureteroscope 101 to at least one of the host machine 168, the computer 172, or the display 170. The cable 174 also may allow the communication interface 166 to communicate with and receive electric power from the host machine 168 or other power sources. The cable 174 also may be configured to allow the communication interface 166 to transmit image data captured at the optoelectronic module 152 to the host machine 168 for processing, storing, and displaying.

The communication interface 166 may contain, for example, one or more of a processor board, a camera board and frame grabber, or a power source. The processor board may be coupled by the cable 174 to the host machine 168 for storage and retrieval of images generated by ureteroscope 101. The communication interface 166 also may be configured to communicate with and receive electric power from the host machine 168 or other power source via the cable 174. The communication interface 166 also may transmit image data captured at the distal end 120 to the host machine 168 for processing, storing, and displaying.

The ureteroscope 101 may include one or more controls 176 positioned at or proximate to the control end 108 of the handpiece 102. The one or more controls 176 may include one or more of a switch, a button, a rotatable knob, a movable tab, and the like. In some embodiments, the one or more controls 176 are configured to adjust views presented on the display 170. For example, the one or more controls may be configured to adjust at least one of a brightness, a zoom, a focus or a contrast of one or more images displayed on the display 170. Accordingly, the one or more controls 176 allow a user to adjust views presented on the display 170 and/or computer 172 according to the user's preference and as necessary during use of the ureteroscope 101. In some embodiments, at least one of the one or more controls 176 is configured to activate (e.g., turn on) or deactivate (e.g., turn off) at least one light source 154 (FIG. 1B) at the distal end 120 of the catheter 104. In some embodiments, at least one of the one or more controls 176 is configured to activate and deactivate the optoelectronic module 152.

The ureteroscope 101 may be operated to perform or complete selected tasks manually, automatically, or a combination thereof. Some ureteroscopic functions may be implemented with the use of components that comprise hardware, software, firmware or combinations thereof. While general-purpose components such as general purpose computers or oscilloscopes may be used in the ureteroscope 101, dedicated or custom components such as circuits, integrated circuits or software may be too. For example, some functions are implemented with a plurality of software instructions executed by one or more data processors, which is part of a general-purpose or custom computer. The one or more data processors may be in at least one of the communication interface 166, the host machine 168, the computer 172, or the display 170. In some embodiments, the data processor or computer comprises volatile memory for storing instructions and/or data and/or a non-volatile storage, for example, a magnetic hard-170k and/or removable media, for storing instructions and/or data. In some embodiments, implementation includes a network connection. In some embodiments, implementation includes a user interface, generally comprising one or more input devices (e.g., allowing input of commands and/or parameters) and output devices (e.g., allowing reporting parameters of operation and results).

Alternatively, the handpiece 102 may further include a compact battery module for supplying power to the optoelectronic module 152 and the at least one light source 154. The power source in the handpiece 102 may be, for example, one or more conventional dry-cell disposable batteries or lithium ion rechargeable batteries.

As previously discussed, the endoscopic system 100 may include a laser 158. The laser 158 is connected to the laser fiber 138 such that a laser emitted by the laser 158 enters the laser fiber 138 and is emitted from the distal portion 140 of the laser fiber 138. The laser 158 may include any suitable laser. In an embodiment, the laser 158 includes a thulium laser (e.g., a thulium fiber laser), a holmium laser (e.g., a holmium:YAG laser), or any other laser. In an embodiment, the laser 158 may be controlled by at least one of the communication interface 166, the host machine 168, the computer 172, or the controls 176.

Other examples of endoscopic systems and endoscopes are disclosed in International Application No. PCT/US19/65616 filed on Dec. 11, 2019, the disclosure of which is incorporated herein, in its entirety, by this reference.

The ureteroscope 101 illustrated in FIGS. 1A-1C is merely one example of a ureteroscope that may be used in any of the endoscopic systems disclosed herein. FIG. 2 is a cross-sectional schematic of a ureteroscope 201, according to an embodiment. Except as otherwise disclosed herein, the ureteroscope 201 is the same or substantially similar to any of the ureteroscopes disclosed herein. For example, the ureteroscope 201 may include a handpiece 202 and a catheter 204 extending from the handpiece 202. The ureteroscope 201 also include a working channel port 224, a working channel (not shown), a laser fiber port 234, a laser fiber channel (not shown) that is distinct from the working channel (not shown), and at least one optional conduit 242 extending from the laser fiber port 234 to the laser fiber channel 236.

At least one of the laser fiber port 234, the laser fiber channel 236, or the optional conduit 242 exhibits a fiber bend 244. As previously discussed, the fiber bend 244 may inhibit insertion of a laser fiber (not shown) into the laser fiber port 234 and through the laser fiber channel 236 and the optional conduit 242. As such, the ureteroscope 201 may include an introducer element 278 located at, on, or around at least a portion of the fiber bend 244. The introducer element 278 is configured to facilitate insertion of the laser fiber through the fiber bend 244.

In an embodiment, the introducer element 278 is formed in or integrally formed with the walls that define at least a portion of the fiber bend 244. In an example, the introducer element 278 includes at least one polished surface that exhibits a surface roughness that is less than the rest of the surface(s) defining at least one of laser fiber port 234, the laser fiber channel 236, or the optional conduit 242. The polished surface of the introducer element 278 decreases the likelihood that the laser fiber becomes caught on the introducer element 278 and reduces the coefficient of friction (e.g., static or kinetic coefficient of friction) between the laser fiber and the introducer element 278 thereby decreasing the likelihood that the laser fiber buckles during insertion. In an example, the introducer element 278 includes a material exhibiting a coefficient of friction (e.g., static or kinetic coefficient of friction) between the laser fiber and the introducer element 278 that is less than the coefficient of friction of between the laser fiber other surface(s) that defines at least one of the laser fiber port 234, the laser fiber channel 236, or the optional conduit 242. The lower coefficient of friction decreases the likelihood that the laser fiber buckles during insertion. In an embodiment, the introducer element 278 includes a material exhibiting a hardness that is greater than another portion of at least one of the laser fiber port 234, the laser fiber channel 236, or the optional conduit 242. The hardness of the introducer element 278 decreases the likelihood that the laser fiber penetrates or otherwise becomes snagged on the introducer element 278 during insertion.

In an embodiment, the introducer element 278 is configured to move thereby preventing or at least inhibiting the laser fiber from becoming caught on the introducer element 278. In an example, the introducer element 278 is configured to rotate around the path that the laser fiber moves along. In an example, the introducer element 278 is configured to translate, such as move in at least substantially the same direction that the laser fiber moves during insertion. In such an example, the introducer element 278 may include a conveyor belt-like mechanism, one or more small roller pins (e.g., exhibit a radius less than 500 μm, less than 250 μm, or less than 100 μm) with the axis of rotation oriented perpendicular to the movement of the laser fiber, or any other mechanism that may move in at least substantially the same direction as the laser fiber.

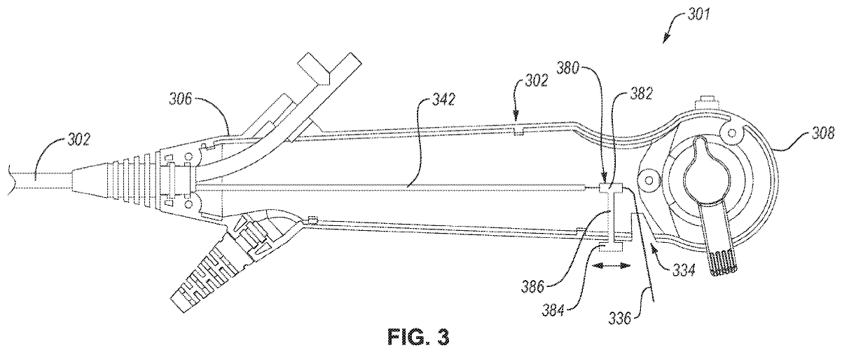

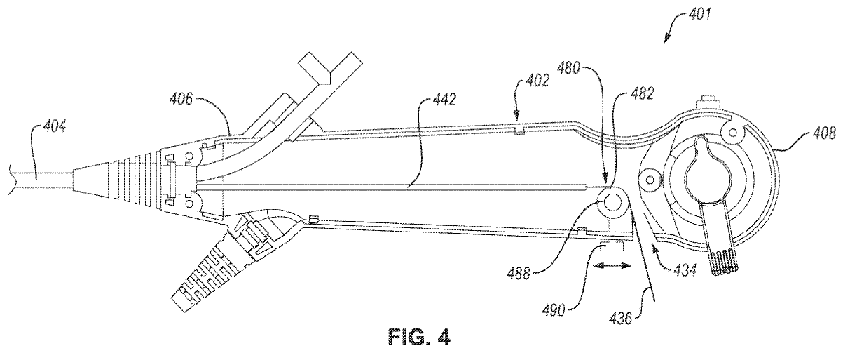

In the ureteroscopes 101, 201 illustrated in FIGS. 1A-2, the laser fibers may move by grabbing the portions of the laser fiber disposed outside of the ureteroscopes and moving the laser fiber relative to the ureteroscopes 101, 201. However, it is noted that the ureteroscopes disclosed herein (e.g., the ureteroscopes illustrated in FIGS. 3 and 4) may include at least one actuator that moves the laser fiber.

FIG. 3 is a cross-sectional view of a ureteroscope 301, according to an embodiment. Except as otherwise disclosed therein the ureteroscope 301 is the same as or substantially similar to any of the ureteroscopes disclosed herein. For example, the ureteroscope 301 includes a handpiece 302 and catheter 304 extending from the handpiece 302. The handpiece 302 defines a laser fiber port 334 and the catheter 302 defines a laser fiber channel (not shown). The handpiece 302 may also include an optional conduit 342.

The handpiece 302 includes an actuator 380. The actuator 380 includes a first portion 382 attached to the laser fiber 338. The first portion 382 may be disposed inside the handpiece 302. The actuator 380 may include a second portion 384 disposed outside of the handpiece 302. The second portion 384 is configured to be manipulated by an user of the ureteroscope 301. The actuator 380 may also include an intermediate portion 386 extending between the first portion 382 and the second portion 384 when the first and second portions 382, 384 are spaced from each other.

The actuator 380 is configured such that manipulation of the second portion 384 by the user moves the laser fiber 338. For example, the actuator 380 is configured such that the second portion 384 may move further or closer to the catheter end 306 of handpiece 302 when the user manipulates the second portion 384. In other words, the second portion 384 may be configured to move generally parallel to a longitudinal axis of the handpiece 302. Moving of the second portion 384 causes a corresponding movement in the first portion 382. For example, in the illustrated embodiment, moving the second portion 384 closer to the catheter end 306 causes the first portion 382 to move closer to the catheter end 306 and moving the second portion 384 further from the catheter end 306 causes the first portion 382 to move further from the catheter end 306. It is noted that the corresponding movement of the first portion 382 relative to the second portion 384 may be reversed (e.g., inverted), for instance, when the first and second portions 382, 384 rotate about a pin. Movement of the first portion 382 causes the laser fiber 338 to move in the same manner. For example, moving the first portion 382 closer to the catheter end 306 causes the portions of the laser fiber 338 attached to the first portion 382 to move closer to the catheter end 306 and moving the first portion 382 further from the catheter end 306 causes t the portions of the laser fiber 338 attached to the first portion 382 to move further from the catheter end 306. Moving the portions of the laser fiber 338 attached to the first portion 382 closer or further from the catheter end 306 causes the portions of the laser fiber 338 to move in the laser fiber channel. For example, moving the laser fiber 338 in the laser fiber channel may cause the distal portion (not shown) of the laser fiber 338 to move relative to the distal end (not shown) of the catheter 304.

In an embodiment, the handpiece 302 may define an opening that allows the actuator 380 to extend between an exterior and interior of the handpiece 302. In the illustrated embodiment, the opening of the handpiece 302 may be configured to have the intermediate portion 386 positioned therein. The opening in the handpiece 302 may be an elongated slot, such as an elongated slot extending in a direction generally extending between the catheter end 306 and the control end 308. Such an elongated slot allows the second portion 384 to move closer or further from the catheter end 306.

FIG. 4 is a cross-sectional view of a ureteroscope 401, according to an embodiment. Except as otherwise disclosed therein the ureteroscope 401 is the same as or substantially similar to any of the ureteroscopes disclosed herein. For example, the ureteroscope 401 includes a handpiece 402 and catheter 404 extending from the handpiece 402. The handpiece 402 defines a laser fiber port 434 and the catheter 404 defines a laser fiber channel (not shown). The handpiece 402 may also include an optional conduit 442.

The handpiece 402 includes an actuator 480. The actuator 480 includes a generally cylindrical portion 488 that is configured to rotation about an axis. The generally cylindrical portion 488 may be configured to receive a portion of the laser fiber 438. For example, the laser fiber 438 may wrap around the generally cylindrical portion 488 such that the generally cylindrical portion 488 operates as a spool. When the generally cylindrical portion 488 is disposed in the interior of the handpiece 402, the actuator 480 may include an lever 490 or one or more other mechanical devices (e.g., gears) coupled to the generally cylindrical portion 488. The lever 490 or other mechanical devices may be positioned on an exterior of the handpiece 402 such that the lever 490 or other mechanical devices may be manipulated by a user. The lever 490 or other mechanical devices may be configured such that manipulation thereof by the user causes the generally cylindrical portion 488 to rotate. Rotating the generally cylindrical portion 488 may cause the generally cylindrical portion 488 to push the laser fiber 438 further towards the catheter end 406 and further into the laser fiber channel or pull the laser fiber 438 from the catheter end 406 and out of the laser fiber channel.

Similar to the handpiece 302 of FIG. 3, the handpiece 402 may define an opening (e.g., elongated slot) for the lever 490 or other mechanical devices may extending to an exterior of the handpiece 402.

It is noted that the actuators 380, 480 illustrated in FIGS. 3 and 4 may be operated using other techniques other than what is illustrated in FIGS. 3 and 4. For example, the first portion 382 and/or the generally cylindrical portion 488 may be attached to a motor and the motor is configured to move the first portion 382 and/or the generally cylindrical portion 488. The motor may be controlled by at least one of the communication interface, the host machine, the computer, or the controls (e.g., the communication interface 166, the host machine 168, the computer 172, or the controls 176 illustrated in FIGS. 1A and 1B). It is noted that the laser fibers of the ureteroscopes disclosed herein may be used with different actuators than the actuators disclosed herein. It is also noted that the actuators of the ureteroscopes disclosed herein may include a locking mechanism to prevent moving of the actuator when the locking mechanism is activated and allow moving the actuator when the locking mechanism is deactivated.

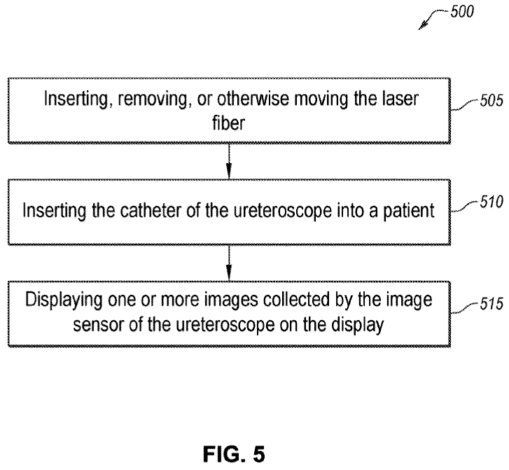

FIG. 5 is a flow diagram of a method 500 of operating any of the ureteroscopes disclosed herein. The method 500 includes an act 505 of inserting, removing, or otherwise moving the laser fiber. The method 500 includes act 510 of inserting the catheter of the ureteroscope into a patient. The method 500 also includes an act 515 of displaying one or more images collected by the image sensor of the ureteroscope on the display.

Acts 505, 510, and 515 of the method 500 are for illustrative purposes. For example, the acts 505, 510, and 515 of the method 500 may be performed in different orders, split into multiple acts, modified, supplemented, or combined. In an embodiment, one or more of the acts 505, 510, and 515 of the method 500 may be omitted from the method 500. Any of the acts 505, 510, and 515 of the method 500 may include using any of the handpieces, ureteroscopes, or the endoscopic systems disclosed herein.

The act 505 of the method 500 includes inserting, removing, or otherwise moving the laser fiber. In an embodiment, act 505 may include inserting the laser fiber into the ureteroscope. For example, the laser fiber may be inserted into the ureteroscope when the laser fiber is not pre-loaded into the ureteroscope, when the laser fiber was previously removed from the ureteroscope, or when a laser fiber that was previously disposed in the ureteroscope is replaced. Inserting the laser fiber into the ureteroscope may include inserting the laser fiber into the laser fiber port defined by the handpiece (e.g., into the funnel structure). Inserting the laser fiber into the ureteroscope may also include threading the laser fiber into the laser fiber channel and the optional conduit until the distal portion of the laser fiber is proximate to the distal end of the catheter. Inserting the laser fiber may include using one or more features disclosed herein (e.g., rotating or translating the introducer element 278 of FIG. 2) to decrease the likelihood that the laser fiber buckles. Inserting the laser fiber may also include attaching the laser fiber to an actuator, such as attaching the laser fiber to the first portion 382 of the actuator 380 of FIG. 3 or wrapping the laser fiber around or otherwise positioning the laser fiber on the generally cylindrical portion 488 of the actuator 480 of FIG. 4.

In an embodiment, act 505 may include removing the laser fiber from the ureteroscope. Act 505 may include removing the laser fiber from the ureteroscope, for example, to increase the flexibility of the catheter, to replace a damaged (e.g., buckled) or contaminated laser fiber, or to use a new laser fiber (e.g., changing the laser connected to the laser fiber may require using a different laser fiber). Removing the laser fiber may include gripping portions of the laser fiber and pulling on such portions of the laser fiber until the laser fiber is removed from the laser fiber channel, from the optional conduit, and out of the laser fiber port. In an example, removing the laser fiber may include detaching the laser fiber from an actuator.

In an embodiment, act 505 may include moving the laser fiber. The laser fiber may be moved for a variety of reasons, such as moving the distal portion of the laser fiber out of the catheter to prevent irradiation of the catheter with the laser emitted from the laser fiber, move the distal portion of the laser fiber closer to an object to be irradiated to minimize dissipation of the laser before the laser irradiates the object, or to move a focal point of the laser onto or otherwise concentrate the energy of the laser on the object to be radiated. Moving the laser fiber may include moving the laser fiber in small increments, such as in increments of about 1 mm or less, about 5 mm or less, about 10 mm or less, about 15 mm or less, or in ranges of about 250 μm to about 750 μm, about 500 μm to about 1 mm, about 750 um to about 1.5 mm, about 1 mm to about 2 mm, about 1.5 mm to about 2.5 mm, about 2 mm to about 3 mm, about 2.5 mm to about 3.5 mm, about 3 mm to about 4 mm, about 3.5 mm to about 4.5 mm, about 4 mm to about 5 mm, about 4.5 mm to about 6 mm, about 5 mm to about 7 mm, about 6 mm to about 8 mm, about 7 mm to about 9 mm, or about 8 mm to about 10 mm. In an example, moving the laser fiber may include gripping the portions of the laser fiber extending out of the handpiece and moving the gripping portions of the laser fiber towards or away from the laser fiber port. In an example, moving the laser fiber may include manipulating the actuator, such as manipulating the second portion 384 illustrated in FIG. 3 or the lever 490 illustrated in FIG. 4. It is noted that the actuators disclosed herein may be effective at controllably moving the laser fiber in small increments.

The act 510 of inserting the catheter of the ureteroscope into a patient may include inserting the distal tip of the catheter into a lumen in the body of human or animal. For example, the catheter may be inserted via a subject's urethra to access various parts of the urinary tract.

The act 515 of displaying one or more images collected by the image sensor of the ureteroscope on the display may include displaying at least one of a still image or a video stream on the display using the one or more images collected by the image sensor. The act 515 also may include displaying two-or three-dimensional reconstructions of the body lumen on the display. The two-or three-dimensional reconstructions of the body lumen may be generated using at least one of a host machine or a computer programmed with image processing software that takes as input the image data output from the image sensor of the ureteroscope.

It is noted that the ureteroscopes disclosed herein may be used in endoscopic procedures that do not involve the urinary tract (e.g., at least one of the urethral, the bladder, the ureter, or the kidney). For example, the ureteroscopes may be used in endoscopic procedures that involve the gastrointestinal tract, the respiratory tract, the ear, the reproductive system, the abdominal or pelvic cavity, the interior of a joint, the organs of the chest, a fetus, the hand, or any other location instead of or in addition to the urinary tract.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. An endoscope, comprising:
a handpiece at least defining a working channel port and a laser fiber port;
a catheter including a proximal end and a distal end opposite the proximal end, the catheter at least defining:
a working channel extending from the proximal end to the distal end of the catheter, the working channel connected to the working channel port;
an optoelectronic module; and
a laser fiber channel distinct from the working channel, the laser fiber channel extending from the proximal end to the distal end of the catheter, the laser fiber channel connected to the laser fiber port; and
a laser fiber;
wherein the working channel, a distal end of the optoelectronic module, and the laser fiber channel are aligned on an axis; and
wherein at least one of:
the laser fiber port includes a funnel structure exhibiting a lateral dimension that decreases with increasing distance from an exterior of the handpiece;
an optional fiber conduit extending from the laser fiber port to the laser fiber channel includes a funnel structure exhibiting a lateral dimension that decreases with increasing distance from an exterior of the handpiece; or
the laser fiber is pre-loaded into the laser fiber channel.

2. The endoscope of claim 1, wherein the laser fiber channel exhibits a diameter of about 50 μm to about 500 μm.

3. The endoscope of claim 1, wherein the laser fiber includes a thulium laser fiber.

4. The endoscope of claim 1, wherein the laser fiber channel exhibits a diameter that is greater than a diameter of the laser fiber by about 50 μm to about 250 μm.

5. The endoscope of claim 3, wherein the laser fiber exhibits a diameter of about 150 μm or less.

6. The endoscope of claim 1, wherein the laser fiber includes a holmium laser fiber.

7. The endoscope of claim 1, wherein at least one of the laser fiber port or the optional conduit extending from the laser fiber port to the laser fiber channel includes the funnel structure exhibiting the lateral dimension that decreases with increasing distance from the exterior of the handpiece.

8. The endoscope of claim 1, wherein the laser fiber port, the laser fiber channel, or the optional conduit includes a fiber bend therein when the catheter is substantially straight.

9. The endoscope of claim 8, wherein the fiber bend exhibits an average radius of curvature that is greater than about 10 cm.

10. The endoscope of claim 8, wherein an average radius of curvature of the fiber bend is greater than a working bend formed in at least one of the working channel port, the working channel, or at least one optional working conduit extending between the working port and the working channel.

11. The endoscope of claim 8, further comprising an introducer element configured to facilitate bending the laser fiber along the bend.

12. The endoscope of claim 11, wherein at least a portion of the introducer element is configured to rotate.

13. The endoscope of claim 11, wherein at least a portion of the introducer element is configured to move in a direction that is generally parallel to at least a portion of the bend.

14. The endoscope of claim 1, wherein the laser fiber is pre-loaded in the laser fiber channel.

15. The endoscope of claim 1, wherein:
the handpiece includes an actuator, the actuator including a generally cylindrical portion disposed within the handpiece, the generally cylindrical portion configured to rotate, the generally cylindrical portion attached to the laser fiber such that rotating the generally cylindrical portion moves the laser fiber.

16. The endoscope of claim 1, wherein:
the handpiece includes an actuator including a first portion attached to the laser fiber and a second portion configured to be manipulated by a user, the second portion attached to the first portion such that manipulation of the second portion causes the first portion to move the laser fiber.

17. The endoscope of claim 1, further comprising a seal configured to at least one of at least partially cover or at least partially fit into at least the laser fiber port.

18. A method of using an endoscope, the method comprising:
inserting a distal end of a catheter of the endoscope into of an individual, the endoscope including:
a handpiece at least defining a working channel port and a laser fiber port;
the catheter including a proximal end and the distal end opposite the proximal end, the catheter at least defining:
a working channel extending from the proximal end to the distal end of the catheter, the working channel connected to the working channel port;
an optoelectronic module; and
a laser fiber channel that is distinct from the working channel, the laser fiber channel extending from the proximal end to the distal end of the catheter, the laser fiber channel connected to the laser fiber port; and
a laser fiber;
wherein the working channel, a distal end of the optoelectronic module, and the laser fiber channel are aligned on an axis; and
wherein at least one of:
at least one of the laser fiber port includes a funnel structure exhibiting a lateral dimension that decreases with increasing distance from an exterior of the handpiece;
an optional fiber conduit extending from the laser fiber port to the laser fiber channel includes a funnel structure exhibiting a lateral dimension that decreases with increasing distance from an exterior of the handpiece; or
the laser fiber is pre-loaded into the laser fiber channel.

19. The method of claim 18, further comprising moving the laser fiber within the laser fiber channel.

20. The method of claim 19, wherein moving the laser fiber within the laser fiber channel includes moving the laser fiber about 5 mm or less.

21. The method of claim 18, wherein moving the laser fiber within the laser fiber channel includes manipulating an actuator.

22. The method of claim 18, further comprising removing the laser fiber from the laser fiber channel.

23. The method of claim 18, further comprising inserting the laser fiber into the laser fiber port and into the laser fiber channel until a distal portion of the laser fiber is at or near the distal end of the catheter.
